(19) United States
(12) Patent Application Publication
(10) Pub. No.: US 2007/0038302 A1
(43) Pub. Date: Feb. 15, 2007

(54) Method and apparatus for the preparation of an inlaid glenoid

(75) Inventors: Jason M. Shultz, Syracuse, IN (US); Brian K. Berelsman, Warsaw, IN (US); Russell Parrott, Winona Lake, IN (US); Nathan A. Winslow, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/204,086

(22) Filed: Aug. 15, 2005

(51) Int. Cl. A61F 2/40; A61B 17/16; A61B 17/17; A61B 17/88

(52) U.S. Cl. 623/19.11; 606/85; 606/96

(57) ABSTRACT

A glenoid component for use in shoulder arthroplasty, the glenoid component is adapted to be implanted into a scapula and engaged by a head of a humeral component. The glenoid component has a body with an exterior periphery which is supported by surrounding bone. The glenoid component further has a bearing surface that is congruent with the natural bearing surface of the adjacent bone. A method of implantation of a glenoid prosthesis is further provided which reduces the damage to adjacent soft tissue materials.

### FIELD OF THE INVENTION

The present invention relates generally to an apparatus and method for shoulder arthroplasty and, more particularly to a glenoid component and other associated surgical components and instruments for use in shoulder arthroplasty.

### BACKGROUND OF THE INVENTION

A natural shoulder joint may undergo degenerative changes due to a variety of etiologies. When these degenerative changes become so far advanced and irreversible, it may ultimately become necessary to replace a natural shoulder joint with a prosthetic shoulder joint. When implantation of such a shoulder joint prosthesis becomes necessary, the natural head portion of the humerus may be resected and a cavity may be created in the intramedullary canal of the host humerus for accepting a humeral component. The humeral component includes a head portion used to replace the natural head of the humerus. Once the humeral component has been implanted, the glenoid cavity positioned at the glenoid may also be resurfaced and shaped to accept a glenoid component. The glenoid component generally includes an articulating surface which is engaged by the head portion of the humeral component.

It is generally known in the art to provide a shoulder joint prosthesis having a glenoid component, as discussed above. However, the current prior art glenoid components along with the associated surgical components and instruments utilized during shoulder arthroplasty may suffer from many disadvantages.

For example, since the glenoid component is subject to various types of loading by the head portion of the humeral component, the glenoid component offers a stable and secure articulating surface. To achieve this, traditional glenoid implantations procedures may result in resecting of most or all of the glenoid soft tissue to prepare a stable base for the implantation of the glenoid component. However, such large resections of the glenoid may exhibit several disadvantages. This resection may remove a significant amount of soft tissue that forms adjacent to the articulating bearing surface with the glenoid cartilage. Additionally, often soft tissue fixation locations such as the teres minor tendon or the bicep brachia tendon may be damaged. Such resections may further increase the amount of labor and complexity of the shoulder arthroplasty.

What is needed then is a glenoid component and associated surgical components for use in shoulder arthroplasty, which do not suffer from the above-mentioned disadvantages. This in turn, will provide a glenoid component which is stable and secure, reduces the overall amount of tissue required to be removed, reduces the overall surgical time and complexity, and enhances and increases attachment strength without increasing overall implant size. It is, therefore, an object of the present invention to provide such a glenoid component and associated surgical components for use in shoulder arthroplasty.

### SUMMARY OF THE INVENTION

In accordance with the teachings of the present invention, an apparatus and method for shoulder arthroplasty is disclosed. The apparatus and method employ a glenoid component and other associated surgical components for use in the shoulder arthroplasty. In this regard, the glenoid component is adapted to be implanted into a scapula at the glenoid fossa or cavity and engaged by a head portion of a humeral component.

In one embodiment, a glenoid component is used for shoulder arthroplasty such that the glenoid component is adapted to be implanted into a scapula and engaged by a head of a humeral component. The glenoid component includes a body having a first articulating surface and a second medial surface which is opposite to the first articulating surface. The first articulating surface is adapted to be engaged by the head of the humeral component and the second medial surface is adapted to be secured to the scapula. A peripheral edge is provided which is adapted to engage a bone forming a cavity in the scapula. The first articulating surface of the glenoid component is congruent with an associated adjacent soft tissue bearing surface.

The present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 represents a view of a glenoid showing the desired resection line;

FIG. 2 represents a view of a glenoid showing a resection profile using first and second circular resections;

FIGS. 3-6 represent a kit of tools used to prepare the resection profile according to the teachings of the present invention;

FIGS. 7A-8B show a partial glenoid prosthesis according to the teachings of the present invention;

FIGS. 9-11 represent the method of implanting the partial glenoid prosthesis;

FIGS. 12 and 13 represent an alternate method of implanting the partial glenoid prosthesis;

FIGS. 14-18 represent an alternate method of implanting a partial prosthetic glenoid;

FIGS. 19 and 20 represent an alternate method of implanting alternate partial prosthetic glenoid;

FIGS. 21-22 represent the use of a partial glenoid resection tool used in an alternate resection method; and

FIGS. 23 and 24 represent the implantation of alternate partial glenoid prosthesis into a prepared glenoid.

### DETAILED DESCRIPTION OF THE FIGURES

The following description of the preferred embodiments is merely exemplary in nature and is in no way intended to limit the invention, its application, or uses.

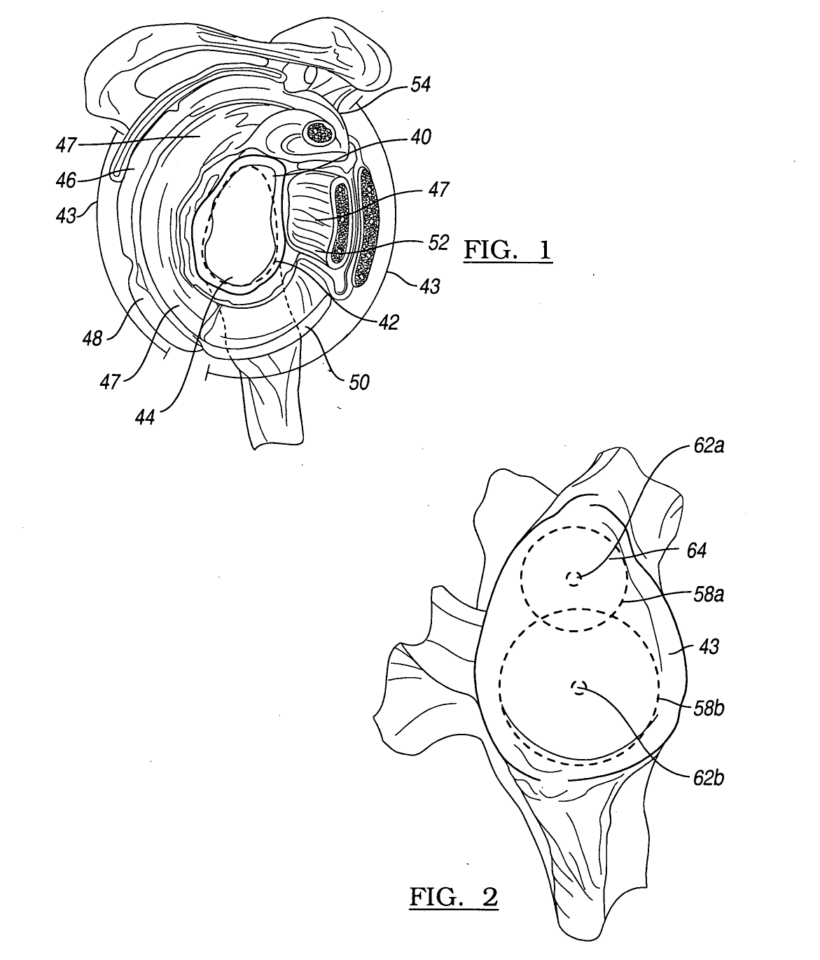

FIG. 1 represents the resection profile 40 for the implantation of a partial resection glenoid 56 according to the teachings of the present invention. The triangular glenoid resection profile 40 is located only in a portion of the glenoid soft tissue 42 immediately adjacent the glenoid cavity 44, and does not affect local adjacent soft tissue structures 43. In doing this, the adjacent soft tissue structures 43, such as glenoid labrum, the infraspinatus tendon 46, teres minor tendon 48, inferior gleno-humeral ligament 50, middle gleno-humeral ligament 52, and bicep brachii tendon 54 can be left untouched if possible. As described below, these soft tissue structures 43 surround the peripheral edge of the partial glenoid implant 56 and function as they would in a normal unaffected joint.

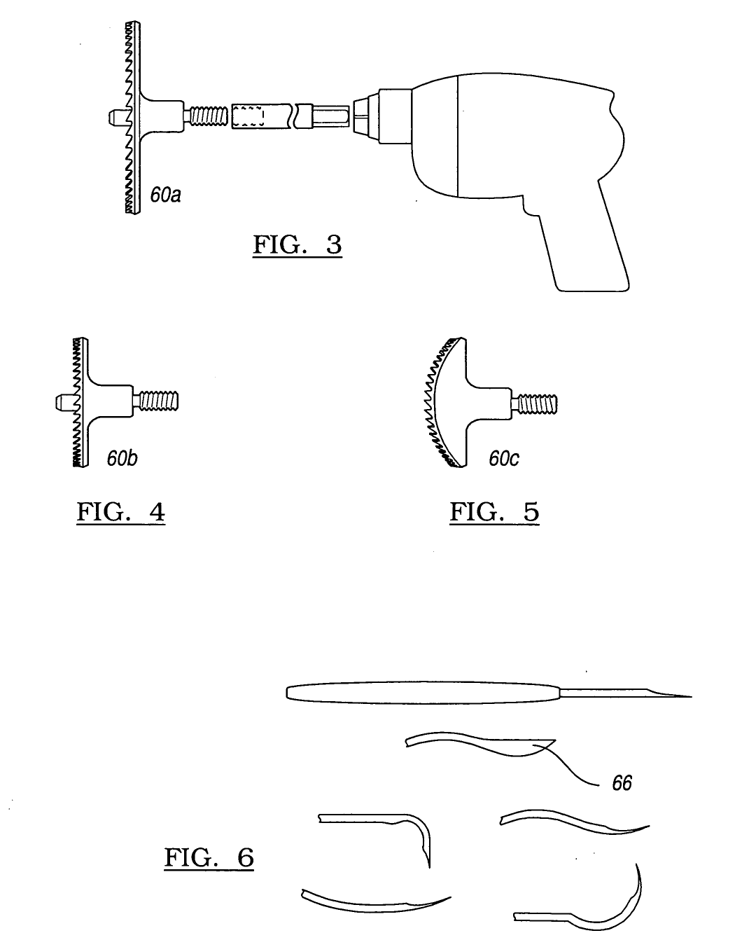

As best seen in FIG. 2, the partial resection of the glenoid begins with a pair of overlapping circular resections 58A and 58B. These resections 58A and 58B are generally centered along a medial axis defining the glenoid cavity. The resections 58A and 58B can occur using flat or spherical cutting tools or rasps 60A-60C as seen in FIGS. 3-5. In this regard, it is envisioned that pilot holes 62A and 62B can be constructed in the non-resected glenoid to act as guides for the circular cutting instruments 60A and 60B and cutting implements described below.

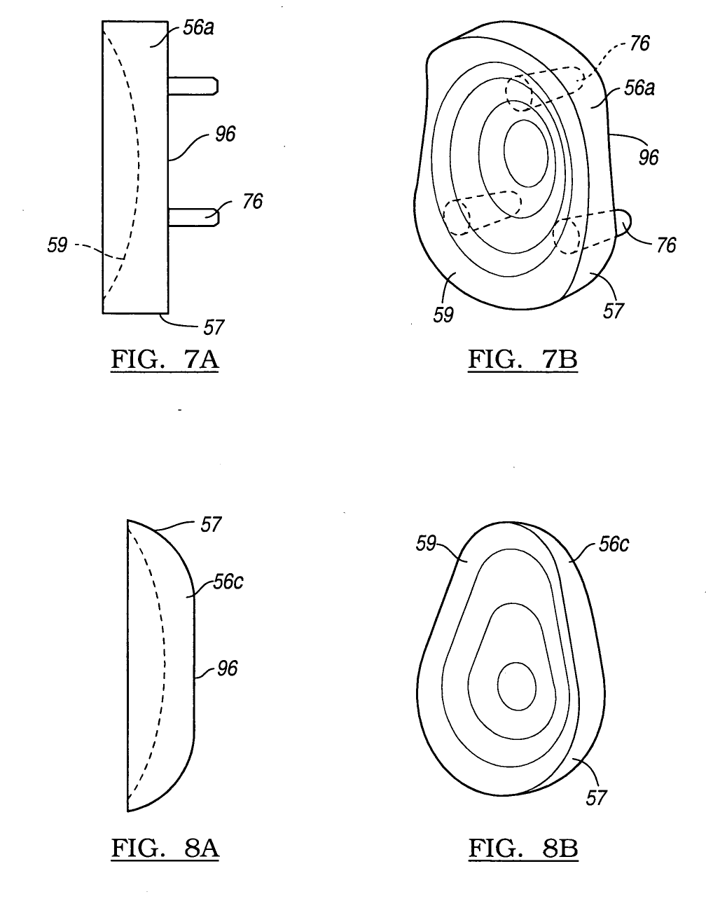

The partial glenoid prosthesis 56A-56C (see FIGS. 7a-8b, 23, and 24) is fixed to the resected portion of the glenoid soft tissue 42. The exterior surface of the partial glenoid 56 is at least partially supported about its peripheral edge 57 by the adjacent bone structures 64. These bone structures 64 define a portion of the natural bearing surface 47 of the resected portion of the glenoid soft tissue cavity 42. The natural bearing surface 47 is congruent or co-planer with the bearing surface 59 of the partial glenoid component.

As seen in FIGS. 7B and 8B, the mating peripheral edge 57 of the implant 56 can be generally triangular to match the resected cavity. This triangular base provides a stable support profile for the glenoid to resection interface. It is envisioned that the outer periphery 57 can vary depending on how much bone is removed.

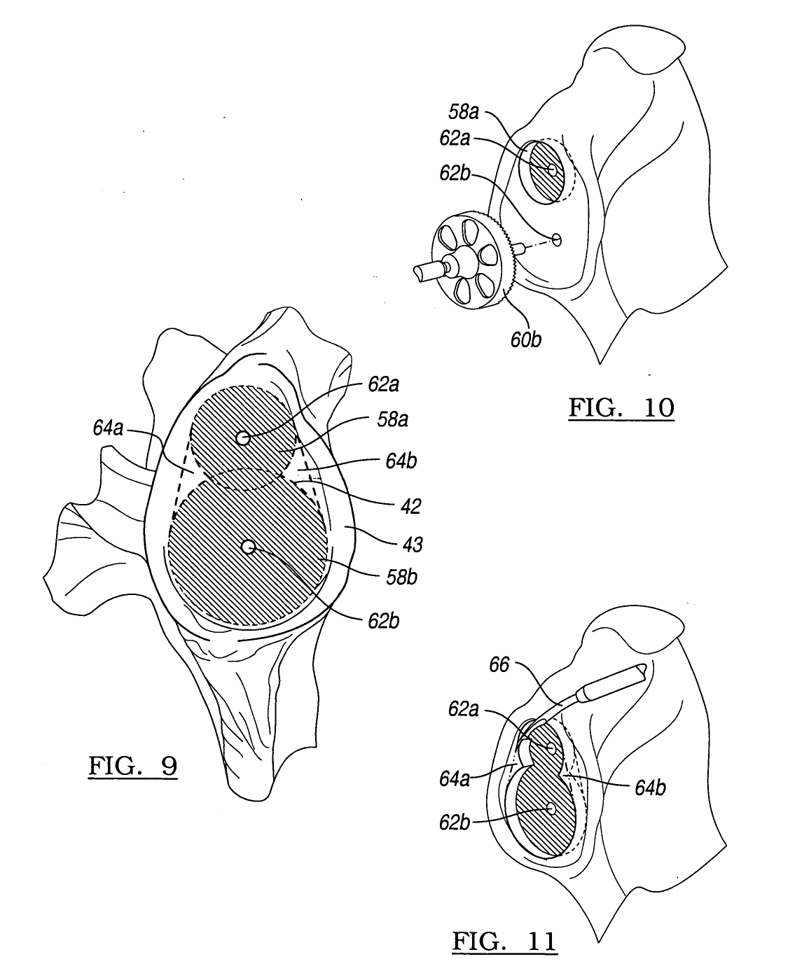

FIGS. 9-11 represent a partial resection of the portion of the glenoid soft tissue cavity 42. As shown in FIG. 9, the cutting tool 60A is used to cut the first circular resection 58A centered about the guide hole 62A. The second resection 58B is then made using the cutting tool 60B centered in guide hole 62B. Optionally, the first and second circular resections 58A and 58B can be partially overlapping to conform to the generally triangular resection profile 40. It should be noted that the circular resections 58A and 58B are generally located within the resection profile 42.

FIG. 10 represents the glenoid after the resections 58A and 58B have been made. Left is the unwanted bone 64A and 64B within the profile, which can be removed prior to the implantation of the partial glenoid implant 56A or 56B. FIG. 11 depicts the removal of the additional bone tissue 64A utilizing a osteotome 66 or similar cutting tool (see FIG. 6).

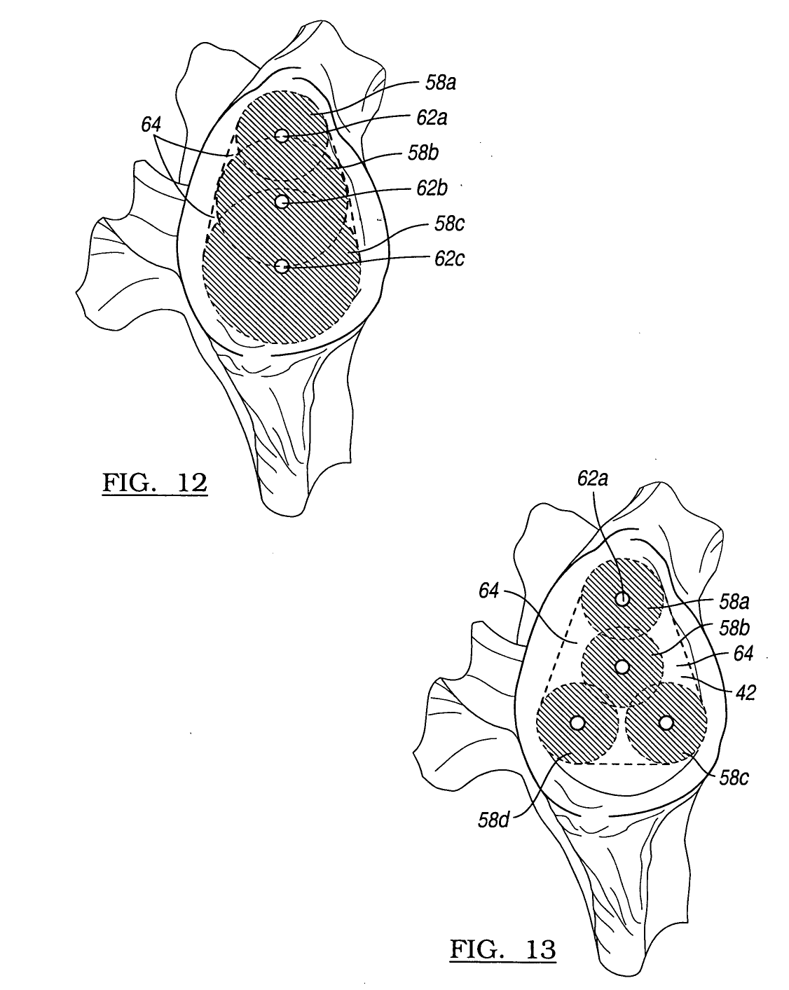

FIGS. 12 and 13 represent alternate methods for preparing the partially resected glenoid. In this regard, these figures show that the bone tissue can be resected utilizing a number of circular resection tools. These circular resection tools can be used to form overlapping or non-overlapping resections within the resection profile 42. As previously described, the additional unwanted bone 64A-64C is preferably removed prior to the implantation of the partial glenoid prosthesis 56.

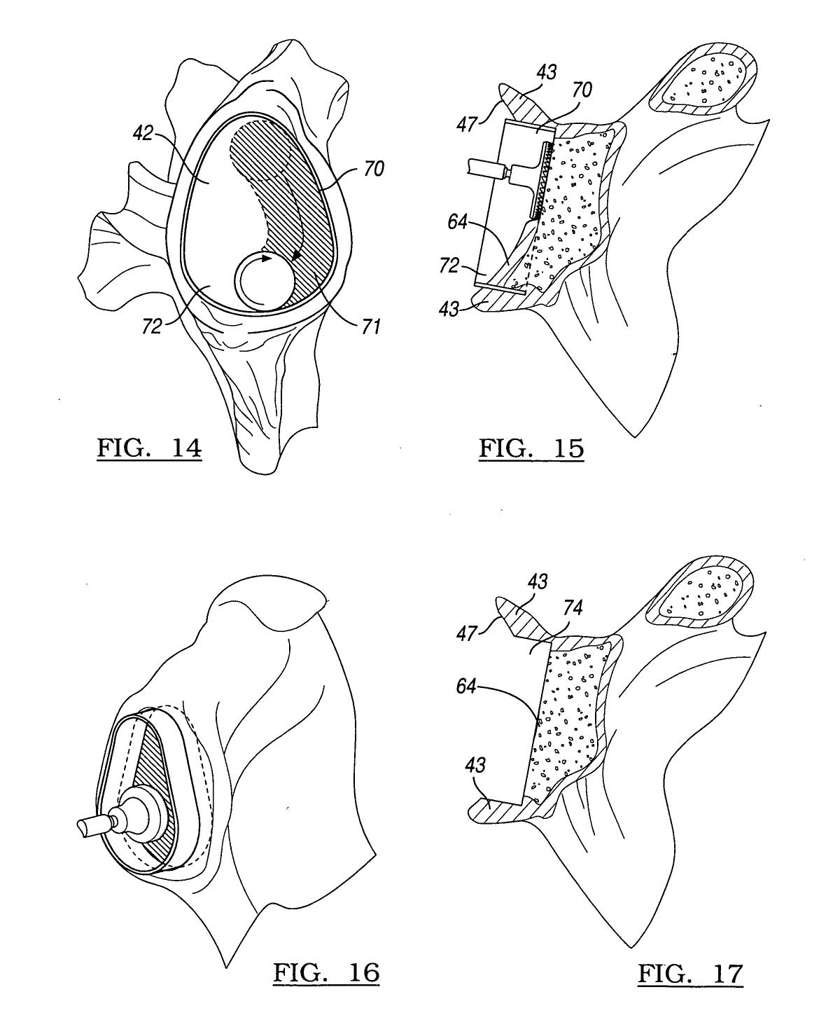

FIGS. 14-17 represent a method of partially resecting a glenoid 42 using a cutting jig 70. The cutting jig 70 is used to guide an appropriate circular cutting tool 60A and can be various shapes, such as a triangle. It is envisioned the cutting jig 70 can have rounded corners 71 having a radius of curvature that is larger or equal to than the radius of the smallest cutting reamer 60A. The cutting jig 70 can be fixed to the glenoid using an alignment pin or can be set into the glenoid using impaction. The cutting tool 60A is rotatably driven and moved about an interior cavity 72 defined by the cutting jig 70 to form a cavity 74.

As shown in FIGS. 19a-21 the implant 56 is positioned within the cavity 74 using minimally invasive procedures. The implant 56 has a coupling surface 75, which is configured to be mated via pegs 76 within the guide holes 62A and 62B formed in the resected glenoid. The implant 56 further has a generally spherical bearing surface 59, which is optionally configured to be congruent with the bearing surface 47 of the associate non-resected and soft tissue component 43.

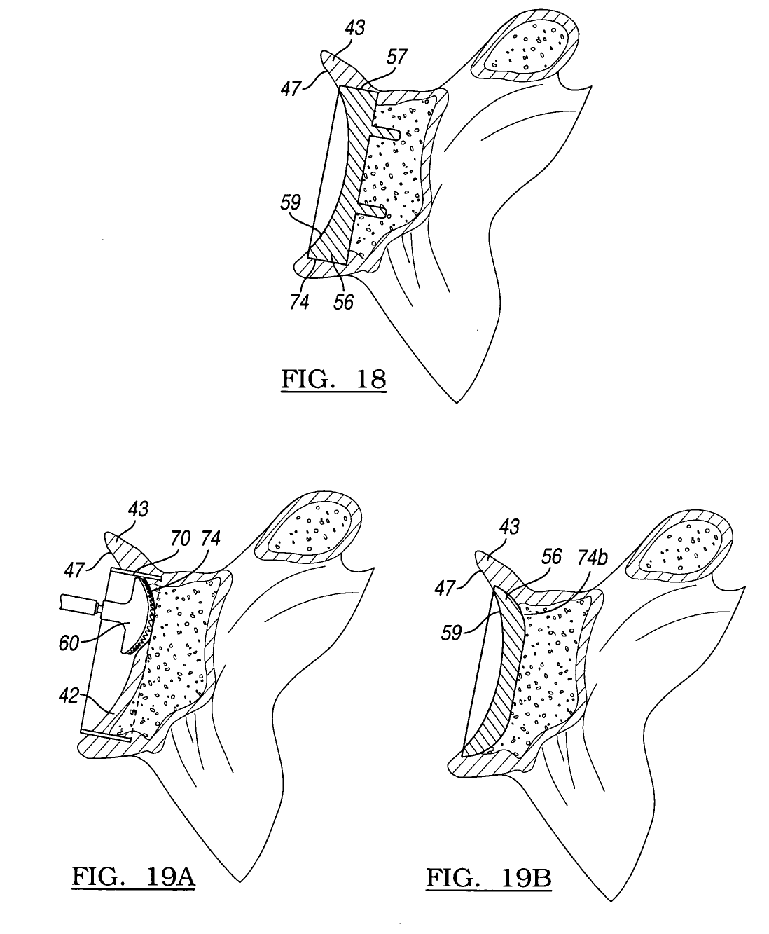

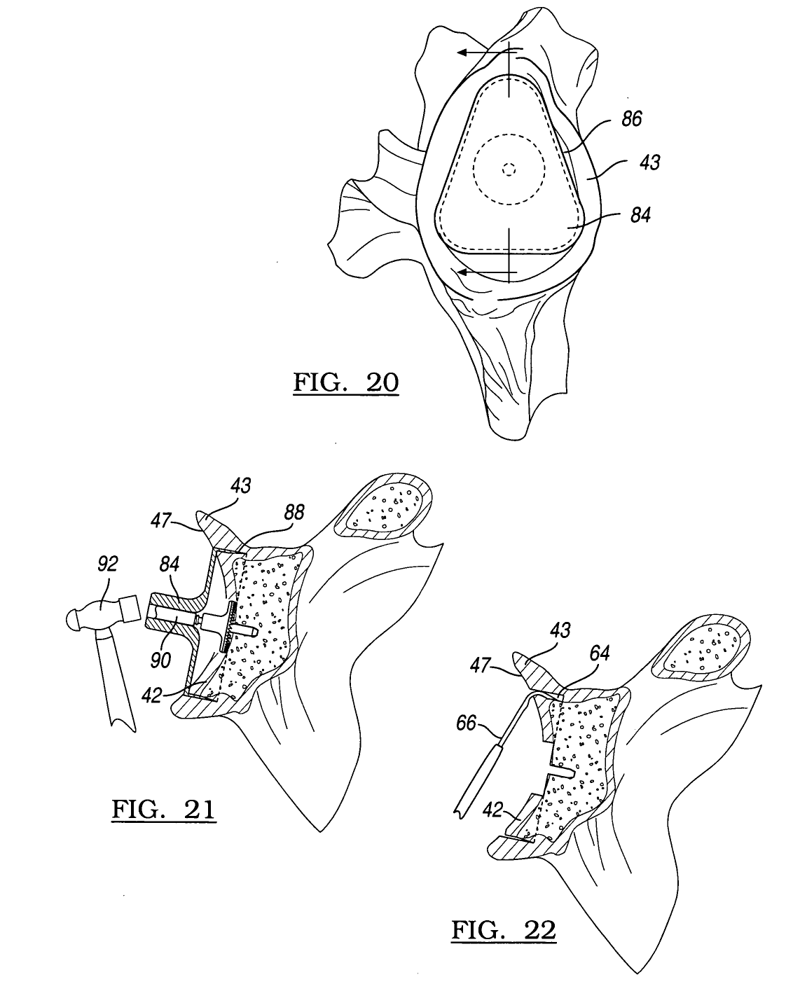

To minimize removal of biological material within the glenoid, it is envisioned that a single spherical reamer 60C can be used to resect adjacent soft tissue structures 43. In this regard, an optional cutting jig 70 can be used. As shown in FIG. 20, this allows the use of an implant 56 having a spherical or curved coupling surface 75. As with the implant 56 shown in FIG. 18, the implant has a congruent bearing surface 59 and pin fixation (not shown).

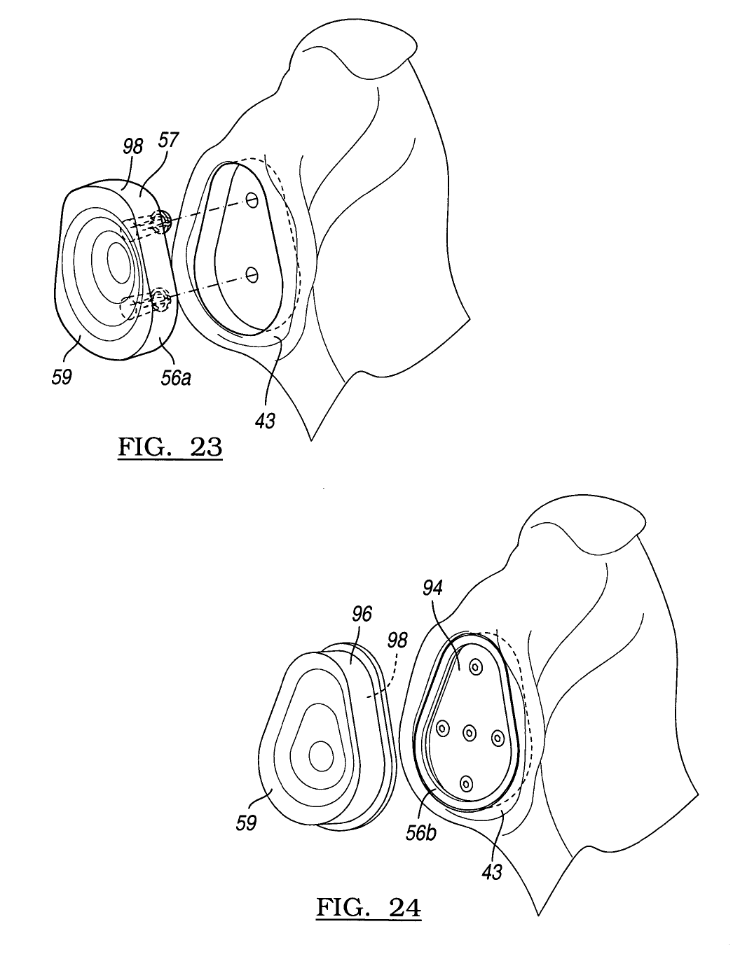

FIGS. 20-22 represent the use of a cutting tool 84 configured to aid in the partial resection of the glenoid. As shown in FIG. 20, the cutting tool 84 has a generally triangular periphery 86, which corresponds to a resection profile 40. The cutting tool 84 has a cutting edge 88 defined on the periphery 86. This cutting edge 88 is used to score the bone to be removed. This score can be used as a visual guide to assist the physician in the removal of the additional unwanted bone 64A.

As shown in FIG. 19, the cutting tool 84 can be centered on a referencing post 90. An impactor 92 is used to set the cutting tool 84 into the glenoid. It is envisioned that the cutting tool 84 can be used in conjunction with the circular cutting tools 60A-60B to help define the resection profile 42. After impaction of the cutting tool 84, the excess bone 64 can be removed using cutting tools, such as the scalpel 66.

FIGS. 23 and 24 represent the implantation of the partial glenoid prosthesis 56A and 56B. The prosthesis 56A and 56B have a generally spherical concave bearing surface 59 configured to interface with the head of a humerus. The generally spherical bearing surface 59 is configured to be positioned congruent to the bearing surface of the adjacent bone 64. The partial glenoid prosthesis 56A and 56B have a coupling surface 98 configured to be mated to the resected glenoid. Prior to the insertion, a drill guide (not shown) can be positioned into the pilot holes to secure the drill guide to the scapula. Holes configured to accept the pegs 76 are drilled into the scapula using a drill template of the drill guide.

The partial glenoid 56A can be directly coupled to the resected surface via coupling pegs or members 76. The coupling pegs 76 can be generally straight or curved and may take the form of a keel. In this regard, the coupling pegs can be coupled to a medial surface 77 at varying angles between about 90° and about 25°. The coupling pegs 76 can be fastened to the partially resected glenoid surface by using cement which has been inserted into a plurality of mounting holes cut into the partially resected glenoid.

Additionally, a modular glenoid 56B can be used. In this regard, a metallic base 94 can be first implanted into the partially resected glenoid cavity 74. A polymer insert 96 is then coupled to the base 94. The polymer insert 96 is configured to have a congruent humeral bearing surface 59 as previously described which is congruent with the bearing surface of the surrounding bone.

The foregoing discussion discloses and describes merely exemplary embodiments of the present invention. One skilled in the art will readily recognize from such discussion and from the accompanying drawings and claims that various changes, modifications, and variations can be made therein without departing from the spirit and scope of the invention. In this regard, it should be understood that the disclosure herein can be used with a total shoulder arthroplasty that replaces a portion of the humerus. Additionally, the prosthesis disclosed herein can be used with a natural humeral joint.

What is claimed is:

1. A glenoid component for use in shoulder arthroplasty, said glenoid component adapted to be implanted into a scapula and engaged by a head of a humerus, said glenoid component comprising:
a body having a first articulating surface and a second medial surface opposite said first articulating surface, said first articulating surface adapted to be engaged by the head of the humeral component and said second medial surface adapted to be secured to the scapula; and
a peripheral edge which is supported about its peripheral edge by the adjacent bone defining a partially resected glenoid cavity.

2. The glenoid component as defined in claim 1 wherein said first articulating surface has a spherical concave surface that is configured to be congruent with a bearing surface defined by the adjacent soft tissue structures.

3. The glenoid component as defined in claim 2 wherein said peripheral edge has a generally triangular shape.

4. The glenoid component as defined in claim 1 wherein said second medial surface has a plurality of fixation members selected from the group consisting of a pegs, a keel, and screws.

5. The glenoid component as defined in claim 1 comprising a base portion configured to be implanted into the partially resected glenoid cavity; and
a polymer insert configured to be coupled to the base.

6. The glenoid component as defined in claim 5 wherein said first polymer insert defines the first articulating surface.

7. A method for implanting a glenoid component during shoulder arthroplasty, said method comprising the steps of:
drilling first and second pilot holes in a glenoid cavity of a scapula;
rotating the first glenoid surface rasp to prepare a first circular resection on the glenoid cavity;
rotating the second glenoid surface rasp to prepare a second circular resection on the glenoid cavity, wherein rotating first and second glenoid surface rasp does not disturb adjacent soft tissue structures;
providing a partial glenoid prosthesis having a body with a first articulating surface and a second medial surface opposite said first articulating surface, said second medial surface adapted to be secured to the first and second circular resections, and a peripheral edge which is at least partially supported by the adjacent bone structure; and
coupling the partial glenoid to the first and second circular resections.

8. The method as defined in claim 7 wherein the first and second circular resections are overlapping.

9. The method as defined in claim 7 further comprising positioning a drill guide adjacent to the scapula; and
drilling a plurality of holes in the scapula using a drill template of the drill guide.

10. The method as defined in claim 7 further comprising positioning a cutting jig adjacent the scapula.

11. A method for implanting a glenoid component during shoulder arthroplasty, said method comprising the steps of:
inserting a first glenoid surface rasp between the bone adjacent the scapula cavity;
rotatably driving the first glenoid surface rasp;
moving the rasp about an interior cavity defined by the adjacent bone to form a partial resection cavity; and
inserting a partial glenoid prosthesis into the partial resection cavity.

12. The method according to claim 11 further comprising positioning a cutting jig within adjacent bone adjacent to a scapula cavity;

13. The method as defined in claim 11 wherein the partial glenoid prosthesis is at least partially supported by the adjacent bone.

14. The method as defined in claim 11 wherein the partial glenoid prosthesis is fully supported about its periphery by the adjacent bone.

15. The method as defined in claim 11 wherein said partial glenoid prosthesis has a first articulating surface and a second medial surface, and fixation pegs extending from said medial surface which are adapted to engage a plurality of mounting holes formed in the scapula.

16. The method as defined in claim 15 further comprising inserting cement between the glenoid cavity and the partial glenoid prosthesis.

17. The method according to claim 15 wherein the partial glenoid prosthesis comprises an first articulating surface configured to interface with a humeral head bearing surface.

18. The method according to claim 11 wherein the partial glenoid prosthesis comprises a generally spherical concave bearing surface.

19. The method according to claim 18 wherein the generally spherical concave bearing surface is congruent to a bearing surface formed by the adjacent bone.

20. The method according to claim 19 wherein the partial glenoid prosthesis is positioned so as to be at least partially supported by the adjacent bone.

21. The method according to claim 20 wherein the body comprises a polymer bearing insert and a base.
